(19) United States
(12) Patent Application Publication
(10) Pub. No.: US 2019/0142512 A1
(43) Pub. Date: May 16, 2019

(54) Devices and Methods to Remove Hair

(71) Applicant: Hong Kong Health and Beauty Limited, Hong Kong (CN)

(72) Inventor: Haiying CHEN, Hong Kong (CN)

(21) Appl. No.: 16/309,919

(22) PCT Filed: Jun. 14, 2017
(86) PCT No.: PCT/CN2017/088260

§ 371 Date: Dec. 13, 2018

Related U.S. Application Data
(60) Provisional application No. 62/350,193, filed on Jun. 15, 2016.

(51) Int. Cl. A61B 18/18; A61N 5/06

(52) U.S. Cl. CPC A61B 18/18; A61B 2018/00476; A61N 5/0617

(57) ABSTRACT

A device to remove unwanted hair on a target area (109) includes a LED unit (201), which comprises at least two types of LEDs to emitting two or more spectrum; a skin type detector (102), which comprises a light source (103) and a photoreceptor (104); and a LED driver (101), which communicates with the skin type detector (102) to sending and receiving signal, and further powers the LED unit (201) with a plurality of separate output channels. A method for removing hair using the device is also provided.

### FIELD OF INVENTION

The present invention relates to devices and methods useful in hair removal. The apparatuses can, for instance, permanently remove unwanted human or animal hair.

### BACKGROUND OF INVENTION

In order to remove unwanted hair, some electronic devices use light, such as intense pulsed light (IPL) or laser light. Electronic devices using IPL to remove hair are often bulky and require a large circuit board and fan. Electronic devices using laser light to remove hair can pose hazards to the user. For example, continuous irradiation can lead to skin redness, blistering, burns, and other side effects. Additionally, laser light emits a monochromatic light that is not suitable for different skin types.

New methods and apparatus for removing unwanted hair are desired.

### SUMMARY OF INVENTION

One example embodiment is an electronic device to remove unwanted hair on a target area. The electronic device includes a LED unit that includes at least two LEDs that emit light with different wavelengths from each other; a skin type detector including a light source and a photoreceptor, that detects a skin type of a target area; and a LED driver that electrically communicates with the skin type detector and transmits power to the LED unit through a plurality of separate output channels. Peak wavelengths of the LEDs range from 600 nm-1200 nm, and each of the two LEDs in the LED unit is independently powered by one of the separate output channels.

Other example embodiments are discussed herein.

### BRIEF DESCRIPTION OF FIGURES

FIG. 1 shows an electronic device for hair removal in accordance with an example embodiment.

FIG. 2 shows a LED unit including three different types of LEDs (L1, L2, and L3) that emit light with different peak wavelengths in accordance with an example embodiment.

FIG. 3a, FIG. 3b, and FIG. 3c show spot shapes of an effective irradiation area in accordance with example embodiments.

FIGS. 4a and 4b show comparisons of overlap areas between square spot shapes with circular spot shapes of the effective irradiation area in accordance with example embodiments.

FIG. 5 shows a schematic diagram of a flexibly wearable part including LED unit in accordance with an example embodiment.

FIG. 6 shows a hair removal process in accordance with an example embodiment.

FIG. 7 shows a table with suggested LED wavelength to be used for different skin types in accordance with an example embodiment.

FIG. 8 shows a test result of an output energy density and an effective irradiation area size of the skin with an LED in accordance with an example embodiment.

FIG. 9a, FIG. 9B, FIG. 9c, and FIG. 9d show a schematic diagram of circuit layouts in accordance with example embodiments.

### DETAILED DESCRIPTION OF THE PREFERRED EMBODIMENTS

As used herein and in the claims, “comprising” means including the following elements but not excluding others.

As used herein and in the claims, “connect” refers to electrical coupling or connection either directly or indirectly via one or more electrical means unless otherwise stated.

An example embodiment includes an electronic device and methods that removes unwanted hair from a person or animal utilizing light emitting diodes (LEDs) as the light source.

LED spectrum has a narrow Full width at half maximum (FWHM) in the range of tens of nanometers. The peak wavelength can be changed by adjusting a band gap energy of the materials forming the p-n junction. LED is low voltage device, and the drive circuit is safe. Pulse interval thereof can also be short, greatly reducing the time of charging so as reducing the waiting time for users. Customer experience is improved.

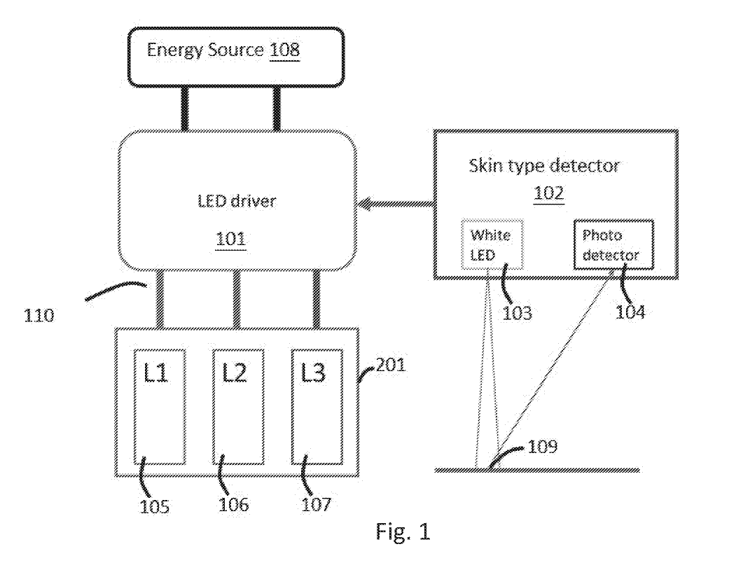

An example embodiment is an electronic hair removal device that includes a LED driver 101, a LED unit 201, and a skin type detector 102, as shown in FIG. 1.

The LED driver 101 electrically connects with the LED unit 201, the skin type detector 102, and an energy or power source 108. The LED driver 101 communicates with the skin type detector 102 to send and receive signals, and further powers the LED unit 201 via a plurality of separate output channels 110.

The LED unit 201 comprises two or more types of LEDs that emit light with different wavelengths or frequencies from the electromagnetic spectrum. For example, L1, L2, and L3 each emit light with a different frequency or wavelength from each other.

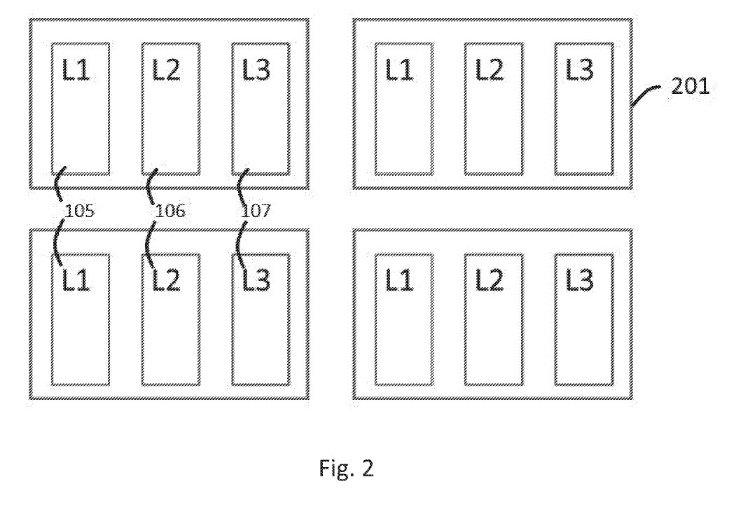

FIG. 2 shows four LED units, one LED unit incorporates three types of LEDs, L1 105, L2 106, and L3 107. Peak wavelengths of the LEDs, according to an example embodiment, range from 600 nanometers (nm) to 1200 nm. In a further example embodiment, the peak wavelength is 694 nm, 790 nm, 808 nm, 810 nm, or 1064 nm. The spectrums of L1 105, L2 106 and L3 107 are different with each other. In a further example embodiment, L1's spectrum peaks at 690 nm, while L2 at 810 nm and L3 at 1064 nm. In another example embodiment, the peak wavelength and numbers of LED could be changed as required.

Each LED L1, L2 or L3 in the LED unit 201 is independently powered by one of the separate output channels. Every output channel is independently controlled and modulated by the LED driver 101 to achieve different combination of light. In one example, two or more LEDs emit light at same portion of the target area.

The skin type detector 102 comprises a light source 103 and a photoreceptor 104 to detect a skin type of a target area 109. In a further example embodiment, the light source is a white light LED.

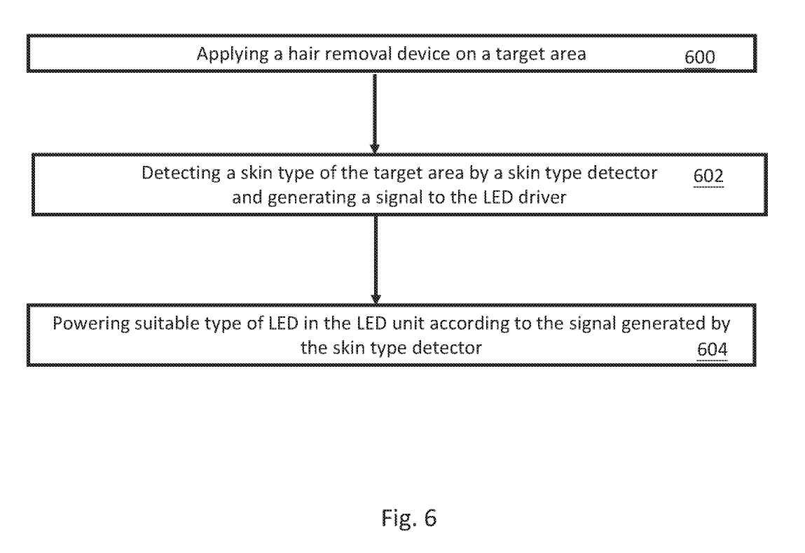

In one example embodiment, a method of removing unwanted human or animal hair using the hair removal device described above is provided as shown in FIG. 6.

Block 600 states applying a hair removal device on a target area 109. The skin type detector 102, upon receiving a signal from the LED driver 101, detects the skin type by illuminating the light source 103 on the skin surface of the target area, and reading its reflection light with the photoreceptor 104 as stated in Block 602. According to the reflected light, the skin type detector 102 generates a signal of the skin type to the LED driver 101. Block 604 states that the LED driver 101, based on the received signal, powers suitable type of LED in the LED unit 201 for following hair removing process to achieve better results and avoid side effects.

The skin types are classified using the Fitzpatrick skin classifications, consisting of type I (pale white), type II (white), type III (cream white), type IV (light brown), type V (brown) and type VI (dark brown). In a further example embodiment, if fairer skin type is detected or determined, LEDs with short wavelength will be turn on and others locked. For darker skin type, only LEDs with longer wavelength will be turned on and others locked. Turning on and off the LEDs in this manner reduces or eliminates redness or even blisters of the skin that are present with conventional electronic hair removal devices.

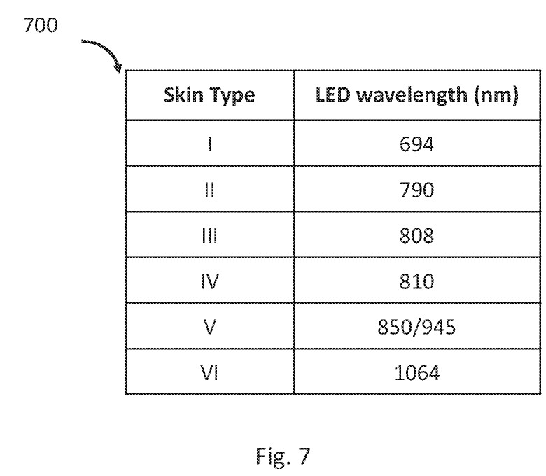

A table 700 in FIG. 7 shows suggested LED wavelength to be used for different skin types. The LEDs emitting light with wavelengths of 694 nm, 790 nm, 808 nm, 810 nm, 850 nm/945 nm or 1064 nm is applied to skin type I, II, III, IV, V and VI, respectively.

In another example embodiment, the LED L1, L2 or L3 is powered with a current pulse of 1-5 Amps (A), a pulse width of 20-400 milliseconds (ms) and an off time of 0.5-10 second (s). In another example, the LED L1, L2 or L3 is powered with a current pulse of 2 A, a pulse width of 390 ms, and an off time of 2 s.

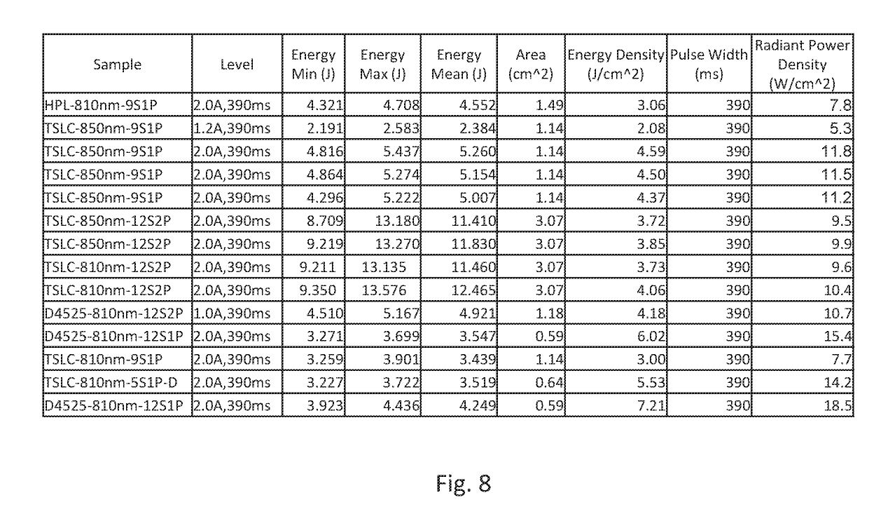

FIG. 8 shows a test result of an output energy density and an effective irradiation area size of the skin with an LED light in accordance with an example embodiment. The results show that the output density and the area size are sufficient for home use hair removal device.

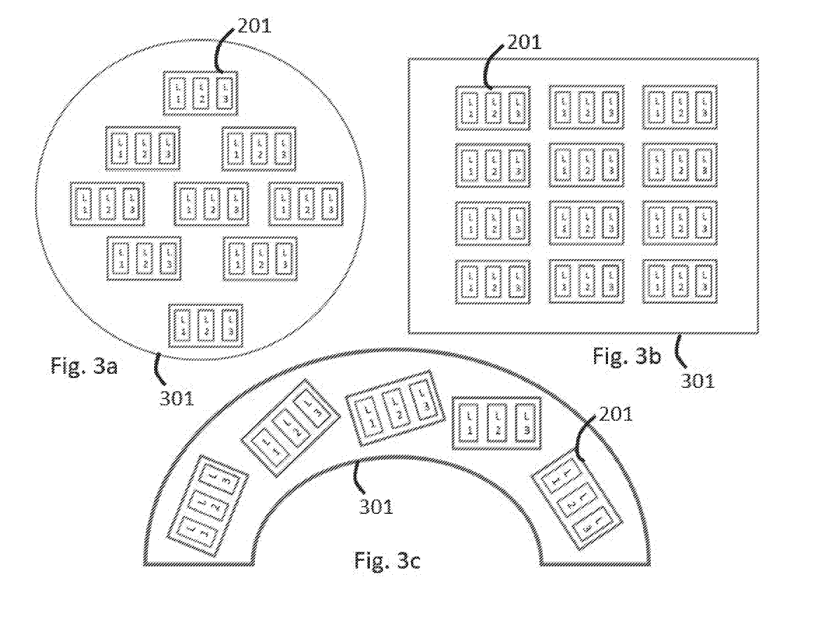

In another example embodiment, a spot of an effective irradiation area 301 of the hair removal device is circular, square, or irregular, in order to achieve a particular location irradiation and hair removal. FIG. 3a, FIG. 3b and FIG. 3c show the spot shape of plural LEDs on a printed circuit board 302 (not shown).

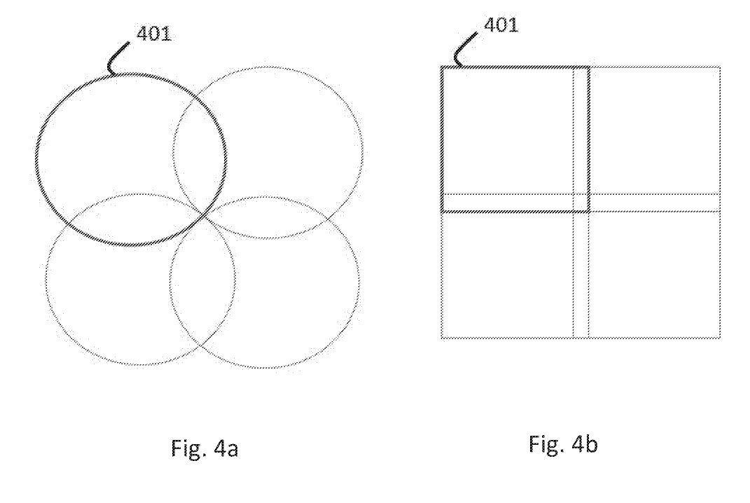

In another example embodiment, LED unit further comprises a lens that can achieve a square or rectangular beam spot shape 401. By this way, an overlap area may be reduced when applying light on a large skin area. This enables an example embodiment to provide quicker treatment process and reducing side effects when compared with conventional electronic hair removal devices. FIG. 4 shows a comparison of overlap areas between square spot shapes with circular spot shapes.

In another example embodiment, LED L1, L2 and L3 are small and are placed on a flexible circuit board. A surface of the LED unit is encapsulated with a flexible material to achieve a deformable light source such that it can be applied on uneven arears of the skin while achieving uniform hair removal results. In a further example embodiment, the flexible material is silicone.

In another example embodiment, LED unit 201 and the skin detector 102 are located adjacent to the LED driver 101.

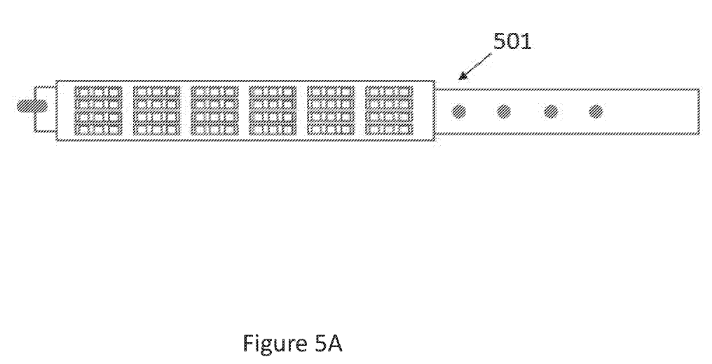

In another example embodiment, LED unit 201 and the skin detector 102 are separated from the LED driver 101 but in electrical communication with it. LED unit is placed on a flexible circuit board and encapsulated with a flexible material such that the LED unit is configured as a flexibly wearable part 501. In a further example embodiment, the flexibly wearable part 501 is in a wristband shape, and can be apply on limbs or other body parts of a person easily and quickly, as shown in FIG. 5.

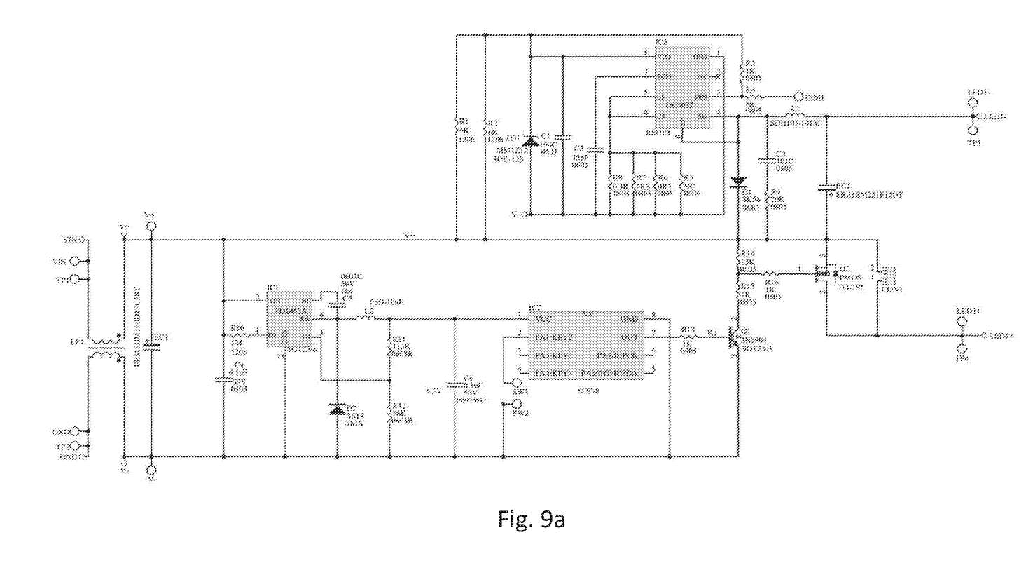

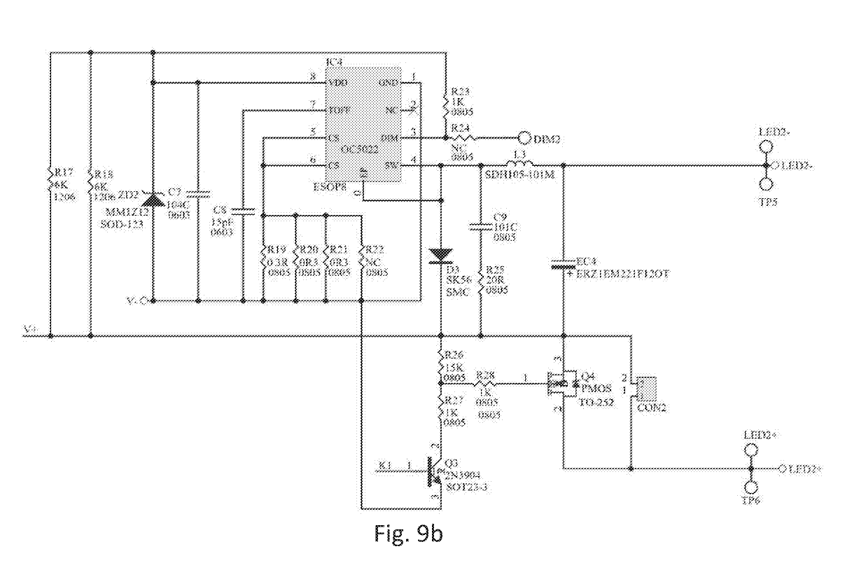

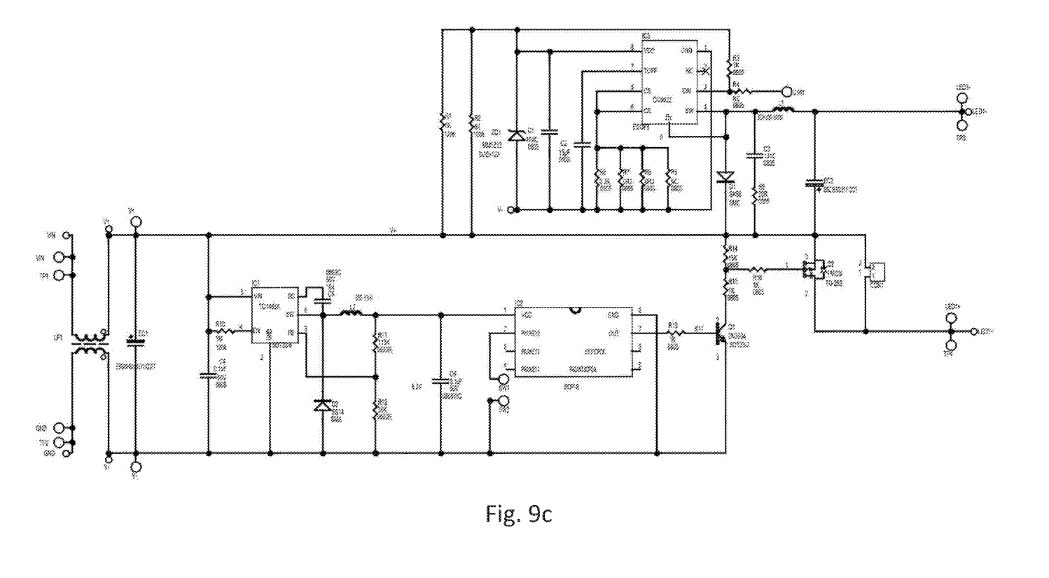

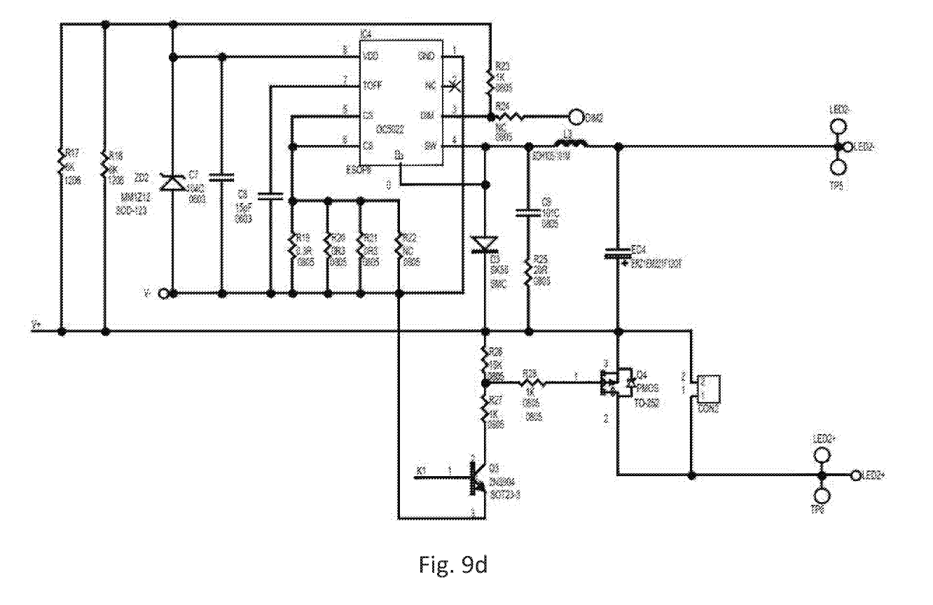

In one example embodiment, a circuit of a constant voltage chip is shown in FIG. 9a to convert an input voltage of 24V into an output voltage of 3.3V for a circuit of controller chip of FIG. 9b. In another example embodiment, the circuit of FIG. 9b modulates constant current chips to power the LED L1, L2 or L3 with a current pulse of 1-5 Amps (A), a pulse width of 20-400 milliseconds (ms) and an off time of 0.5-10 second (s). Circuits of constant current chips are shown in FIG. 9c and FIG. 9d.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

What is claimed is:

1. An electronic device to remove unwanted hair on a target area, comprising:
a LED unit that includes at least two LEDs that emit light with different wavelengths from each other;
a skin type detector including a light source and a photoreceptor, that detects a skin type of a target area; and
a LED driver that electrically communicates with the skin type detector and transmits power to the LED unit through a plurality of separate output channels,
wherein peak wavelengths of the LEDs range from 600 nm-1200 nm, and each of the two LEDs in the LED unit is independently powered by one of the separate output channels.

2. The electronic device according to claim 1, wherein the LED unit includes three types of LEDs that each emit light with different wavelengths from each other.

3. The electronic device according to claim 1, wherein the LED unit includes three LEDs, and each of the three LEDs emit a different wavelength that corresponds to a different skin type.

4. The electronic device according to claim 1, wherein the LED unit emits light with wavelengths of 694 nm, 790 nm, 808 nm, 810 nm, 850 nm/945 nm or 1064 nm.

5. The electronic device according to claim 4, wherein the skin types are classified using Fitzpatrick skin classifications, and the LEDs emitting light with wavelengths of 694 nm, 790 nm, 808 nm, 810 nm, 850 nm/945 nm or 1064 nm is applied to skin type I, II, III, IV, V and VI, respectively.

6. The electronic device according to claim 1, wherein the skin type detector detects a skin type of the target area and generates a signal to the LED driver.

7. The electronic device according to claim 6, wherein based on the received signal, the LED driver powers suitable type of LED in the LED unit.

8. The electronic device according to claim 1, wherein the two LEDs are powered with a current pulse of 1 A-5 A, a pulse width of 20 ms-400 ms, and an off time period of 0.5-10 second (s).

9. The electronic device according to claim 1, wherein a spot of an effective irradiation area of the device is circular, square, or irregular.

10. The electronic device according to claim 1, wherein the LED unit is encapsulated with a flexible material to make the LED unit deformable.

11. The electronic device according to claim 1, wherein the LED unit is flexible and wearable on a person.

12. The electronic device according to claim 1, wherein the LED unit is shaped as a wearable wristband.

13. A method of removing hair using the electronic device of claim 1, comprising:
applying light emitted from the electronic device on a target area;
detecting, with the skin type detector, a skin type of the target area;
generating, with the electronic device and based on the skin type detected, a signal to the LED driver; and
powering, by the LED driver, LEDs in the LED unit according to the signal generated by the skin type detector.

14. The method of claim 13, wherein the skin type detector, upon receiving a signal from the LED driver, detects the skin type by illuminating a light source on a skin surface of the target area, and reading reflection light with a photoreceptor in the electronic device.

15. The method of claim 13, wherein the skin types are classified using Fitzpatrick skin classifications, and the LEDs emitting light with wavelengths of 694 nm, 790 nm, 808 nm, 810 nm, 850 nm/945 nm or 1064 nm is applied to skin type I, II, III, IV, V and VI, respectively.
